(19) United States
(12) Patent Application Publication
(10) Pub. No.: US 2008/0233619 A1
(43) Pub. Date: Sep. 25, 2008

(54) Process For the Enantioselective Enzymatic Reduction of Keto Compounds

(75) Inventors: Antje Gupta, Wiesbaden-Medenbach (DE); Anke Tschentscher, Eltville-Hattenheim (DE); Maria Bobkova, Idstein (DE)

Correspondence Address: WORKMAN NYDEGGER, 60 EAST SOUTH TEMPLE, 1000 EAGLE GATE TOWER, SALT LAKE CITY, UT, 84111, US

(73) Assignee: IEP GMBH, Wiesbaden (DE)

(21) Appl. No.: 12/067,752

(22) PCT Filed: Jul. 27, 2006
(86) PCT No.: PCT/EP2006/007425

§ 371 Date: Apr. 21, 2008

(30) Foreign Application Priority Data
Sep. 23, 2005 (AT) A 1570/2005

(51) Int. Cl. C12P 7/00

(52) U.S. Cl. 435/132

(57) ABSTRACT

The present invention relates to a process for the enantioselective enzymatic reduction of keto compounds which is carried out in two phases and uses 4-methyl-2-pentanol, 5-methyl-2-hexanol and/or 2-heptanol for coenzyme regeneration.

The present invention relates to a process for the enantioselective enzymatic reduction of keto compounds with carbonyl reductases.

Carbonyl reductases (further names: alcohol dehydrogenases, oxidoreductases) are known as catalysts for the reduction of carbonyl compounds and for the oxidation of secondary alcohols, respectively. Those enzymes require a coenzyme, for instance, NAD(P)H. The reduction of ketones with the carbonyl reductase obtained from Lactobacillus kefir and with the coenzyme NADPH is known, for example, from U.S. Pat. No. 5,342,767. By means of these enzymes, it is possible to reduce keto compounds to optically active hydroxy compounds. A further process is known, for example, from WO 03/078615.

Optically active hydroxy compounds are valuable chiral components with broad applicability for the synthesis of pharmacologically active compounds, aromatic substances, pheromones, agricultural chemicals and enzyme inhibitors. Thereby, an increasing demand for chiral compounds and thus chiral synthesis technologies can be noted particularly in the pharmaceutical industry, since, in the future, racemic compounds will hardly be used as pharmaceutical preparations.

The asymmetric reduction of prochiral keto compounds is a sector of stereoselective catalysis, wherein biocatalysis constitutes a powerful competitive technology versus chemical catalysis. The chemical asymmetric hydration requires the use of highly toxic and environmentally harmful heavy metal catalysts, of extreme and thus energy-intensive reaction conditions and of large amounts of organic solvents. Furthermore, those methods are often characterized by side reactions and insufficient enantiomeric excesses.

In nature, reductions of prochiral keto compounds to hydroxy compounds and vice versa occur in numerous biochemical pathways, both in the primary metabolism and in the secondary metabolism, in every organism and are catalyzed by different types of secondary alcohol dehydrogenases and oxidoreductases. Normally, these enzymes are cofactor-dependent.

The basic feasibility of using biocatalysts for the reduction of prochiral keto compounds to chiral hydroxy compounds was repeatedly demonstrated in the past on the basis of model systems, wherein both isolated oxidoreductases and various whole-cell biotransformation systems were used for the task. The biocatalytic approach is advantageous with regard to mild reaction conditions, lack of byproducts and often significantly better achievable enantiomeric excesses. The use of isolated enzymes is thereby advantageous in comparison to methods involving whole cells with regard to the achievable enantiomeric excess, the formation of degradation products and byproducts as well as the product isolation. Moreover, the use of whole-cell processes is not possible for every chemical company, since specific equipment and know-how is required therefor.

Recently, it has been possible to demonstrate that the use of isolated oxidoreductases in aqueous/organic two-phase systems with organic solvents is extremely efficient and feasible also at high concentrations (>5%). In the described systems, the keto compound to be reduced, which usually is poorly water-soluble, forms the organic phase together with the organic solvent. Also, the organic solvent itself can partly be dispensed with, the organic phase is then formed from the keto compound to be reduced (DE10119274, DE10327454.4, DE 103 37 401.9, DE 103 00 335.5). Coenzyme regeneration is thereby realized by the simultaneous oxidation of secondary alcohols, for which, in most cases, the inexpensive water-miscible 2-propanol is used.

Examples of suitable R- and S-specific oxidoreductases and dehydrogenases of high enantioselectivity are:

Carbonyl reductase from Candida parapsilosis (CPCR) (U.S. Pat. No. 5,523,223 and U.S. Pat. No. 5,763,236, (Enzyme Microb Technol. 1993 Nov; 15(11):950-8)) or Pichia capsulata ADH(DE10327454.4);

Carbonyl reductase from Rhodococcus erythropolis (RECR) (U.S. Pat. No. 5,523,223), Norcardia fusca (Biosci. Biotechnol. Biochem., 63 (10) (1999), pages 1721-1729), (Appl Microbiol Biotechnol. 2003 September; 62(4):380-6. Epub 2003 Apr. 26), and Rhodococcus ruber (J Org Chem. 2003 Jan 24;68(2):402-6.);

and

R-specific secondary alcohol dehydrogenases from organisms of the genus Lactobacillus (Lactobacillus kefir (U.S. Pat. No. 5,200,335), Lactobacillus brevis (DE 19610984 A1) (Acta Crystallogr D Biol Crystallogr. 2000 December; 56 Pt 12:1696-8), Lactobacillus minor (DE1019274) or Pseudomonas (U.S. Pat. No. 5,385,833)(Appl Microbiol Biotechnol. 2002 August; 59(4-5):483-7. Epub 2002 Jun. 26., J. Org. Chem. 1992, 57, 1532);

In the prior art methods, there exists a demand for improving and simplifying, respectively, coenzyme regeneration. Most alcohol dehydrogenases and oxidoreductases are quickly inactivated at a propanol concentration of >15% by volume, which leads to the result that the latter is not applicable in batch processes with an arbitrary excess relative to the keto compound, whereby, with an equal concentration of keto compound, only unsatisfactory conversions can be achieved with substrates exhibiting an adverse state of equilibrium.

It is the object of the invention to eliminate said disadvantage.

The process according to the invention for the enantioselective enzymatic reduction of keto compounds of general Formula I

R₁—C(O)—R₂ (I)

wherein R1 stands for one of the moieties

1) —(C₁—C₂₀)-alkyl, wherein alkyl is linear-chain or branched,

2) —(C₂—C₂₀)-alkenyl, wherein alkenyl is linear-chain or branched and optionally contains up to four double bonds,

3) —(C₂—C₂₀)-alkynyl, wherein alkynyl is linear-chain or branched and optionally contains up to four triple bonds,

4) —(C₆—C₁₄)-aryl,

5) —(C₁—C₈)-alkyl-(C₆—C₁₄)-aryl,

6) —(C₅—C₁₄)-heterocycle which is unsubstituted or substituted one, two or three times by —OH, halogen, —NO₂ and/or —NH₂, or

7) —(C₃—C₇)-cycloalkyl,

wherein the moieties mentioned above under 1) to 7) are unsubstituted or substituted one, two or three times, independently of each other, by —OH, halogen, —NO₂ and/or —NH₂,
and R₂ stands for one of the moieties

8) —(C₁—C₆)-alkyl, wherein alkyl is linear-chain or branched,

9) —(C₂—C₆)-alkenyl, wherein alkenyl is linear-chain or branched and optionally contains up to three double bonds,

10) —(C₂—C₆)-alkynyl, wherein alkynyl is linear-chain or branched and optionally contains two triple bonds, or

11) —(C₁—C₁₀)-alkyl-C(O)—O—(C₁—C₆)-alkyl, wherein alkyl is linear or branched and is unsubstituted or substituted one, two or three times by —OH, halogen, —NO₂ and/or —NH₂,

wherein the moieties mentioned above under 8) to 11) are unsubstituted or substituted one, two or three times, independently of each other, by —OH, halogen, —NO₂ and/or —NH₂,
is characterized in that
a liquid, two-phase mixture containing

(a) at least 5% by weight/by volume of a compound of Formula (I),

(b) at least 10% by volume of 4-methyl-2-pentanol, 5-methyl-2-hexanol and/or 2-heptanol and

(c) water,

is treated with an oxidoreductase in the presence of a cofactor in order to form a chiral hydroxy compound of general Formula II

R₁—CH(OH)—R₂ (II)

wherein R₁ and R₂ have the above-indicated meanings.

The invention is based on the realization that processes using highly expressed isolated alcohol dehydrogenases and oxidoreductases can be significantly improved and simplified, respectively, by using 4-methyl-2-pentanol, 5-methyl-2-hexanol and/or 2-heptanol not miscible with water for the coenzyme regeneration of NAD(P)H.

Preferred variants of the process according to the invention are characterized in that the liquid, two-phase mixture contains at least 40% by volume, particularly between 40 and 80% by volume, of 4-methyl-2-pentanol, 5-methyl-2-hexanol and/or 2-heptanol, based on the total volume of the reaction batch, if an oxidoreductase of a microbial origin is used.

In the process according to the invention, the reduction of the keto compound is thus carried out in a two-phase system consisting of an aqueous phase containing the cofactor NADH or NADPH and the oxidoreductase and an organic phase formed by the cosubstrate 4-methyl-2-pentanol and the keto compound largely dissolved therein.

The coenzyme regeneration of NAD(P)H is thereby effected by oxidation of the cosubstrate 4-methyl-2-pentanol, 5-methyl-2-hexanol and/or 2-heptanol, which simultaneously serves as a solvent and as an extracting agent particularly for poorly water-soluble keto compounds.

By using 4-methyl-2-pentanol, 5-methyl-2-hexanol and/or 2-heptanol as a solvent and cosubstrate, good conversions (>90%), high concentrations as well as significantly shorter reaction times can also be realized for substrates exhibiting an adverse state of equilibrium.

The above-described process is particularly advantageous also for the reduction of ketones with low boiling points, such as, e.g., 1,1,1-trifluoroacetone, and of those in which the resulting chiral alcohols have boiling points which are below that of water, such as in case of 1,1,1-trifluoropropane-2-ol. In those cases, the separation of hydroxy compounds, acetone, 2-propanol and water by distillation is often hampered.

Furthermore, the alcohols used according to the invention have proven to be stabilizing for many oxidoreductases that are being used, generally resulting in a reduced enzyme consumption in comparison to other aqueous-organic two-phase systems.

Coenzyme regeneration can thereby occur in a substrate-coupled (i.e., one enzyme for the reduction of the keto substrate and for the oxidation of the 4-methyl-2-pentanol) or in an enzyme-coupled manner. In the enzyme-coupled approach, the regeneration of the cofactor NADH or NADPH is effected by means of a second highly expressed isolated secondary alcohol dehydrogenase.

By this method, ttn's (total turn over number, mole of product formed per mole of cofactor) in the range of 10³-10⁶ are achieved. For the most part, the feasible substrate concentrations are thereby significantly above 5% (percentage by volume).

The concentration of the cosubstrate ranges from 10 to 90% by volume of the reaction mixture, preferably between 40 and 80% by volume.

The enzyrne consumption of the oxidoreductase ranges from 10 000-10 Mio U/kg (no upper limit) of the keto compound to be converted. Thereby, the enzyme unit 1 U corresponds to the enzyme amount which is required for reacting 1 μmol of the compound of Formula I per minute (min).

By the term “NADH”, reduced nicotinamide adenine dinucleotide is understood. By the term “NAD”, nicotinamide adenine dinucleotide is understood. By the term “NADPH”, reduced nicotinamide adenine dinucleotide phosphate is understood. By the term “NADP”, nicotinamide adenine dinucleotide phosphate is understood.

By the term chiral “hydroxy compound”, compounds of Formula II

R¹—C(OH)—R2 (II)

are understood, for example, wherein R1 and R2 have the same meanings as in Formula I.

By the term aryl, aromatic carbon moieties comprising 6 to 14 carbon atoms within the ring are understood. —(C₆—C₁₄)-aryl moieties are, for example, phenyl, naphthyl, e.g., 1-naphthyl, 2-naphthyl, biphenylyl, e.g., 2-biphenylyl, 3-biphenylyl and 4-biphenylyl, anthryl or fluorenyl. Biphenylyl moieties, naphthyl moieties and in particular phenyl moieties are preferred aryl moieties. By the term “halogen”, an element from the family of fluorine, chlorine, bromine or iodine is understood. By the term “—(C₁—C₂₀)-alkyl, a hydrocarbon moiety is understood, the carbon chain of which is linear-chain or branched and comprises 1 to 20 carbon atoms, for example, methyl, ethyl, propyl, isopropyl, butyl, tertiary butyl, pentyl, hexyl, heptyl, octyl, nonenyl or decanyl. By the term “—C₀-alkyl”, a covalent bond is understood. By the term “—(C₃—C₇)-cycloalkyl”, cyclic hydrocarbon moieties such as cyclopropyl, cylobutyl, cyclopentyl, cyclohexyl or cycloheptyl are understood. The term “—(C₅—C₁₄)-heterocycle” stands for a monocyclic or bicyclic 5-membered to 14-membered heterocyclic ring which is partially or completely saturated. N, O and S are examples of heteroatoms. Examples for the terms —(C₅—C₁₄)-heterocycle are moieties derived from pyrrole, furan, thiophene, imidazole, pyrazole, oxazole, isoxazole, thiazole, isothiazole, tetrazole, 1,2,3,5-oxathiadiazole-2-oxide, triazolone, oxadiazolone, isoxazolone, oxadiazolidinedione, triazoles, which are substituted by F, —CN, —CF₃ or —C(O)—O—(C₁—C₄)-alkyl, 3-hydroxypyrro-2,4-dione, 5-oxo-1,2,4-thiadiazole, pyridine, pyrazine, pyrimidine, indole, isoindole, indazole, phthalazine, quinoline, isoquinoline, quinoxaline, quinazoline, cinnoline, carboline- and benz-anellated, cyclopenta-, cyclohexa- or cyclohepta-anellated derivatives of said heterocycles. The moieties 2- or 3-pyrrolyl, phenylpyrrolyl such as 4- or 5-phenyl-2-pyrrolyl, 2-furyl, 2-thienyl, 4-imidazolyl, methylimidazolyl, e.g., 1-methyl-2-, -4- or -5-imidazolyl, 1,3-thiazole-2-yl, 2-pyridyl, 3-pyridyl, 4-pyridyl, 2-, 3- or 4-pyridyl-N-oxide, 2-pyrazinyl, 2-, 4- or 5-pyrimidinyl, 2-, 3- or 5-indolyl, substituted 2-indolyl, e.g., 1- methyl, 5-methyl, 5-methoxy-, 5-benzyloxy-, 5-chloro- or 4,5-dimethyl-2-indolyl, 1-benzyl-2- or -3-indolyl, 4,5,6,7-tetrahydro-2-indolyl, cyclohepta[b]-5-pyrrolyl, 2-, 3- or 4-quinolyl, 1-, 3- or 4-isoquinolyl, 1-oxo-1,2-dihydro-3-isoquinolyl, 2-quinoxalinyl, 2-benzofuranyl, 2-benzo-thienyl, 2-benzoxazolyl or benzothiazolyl or dihydropyrinidyl, pyrrolidinyl, e.g., 2- or 3-(N-methylpyrrolidinyl), piperazinyl, morpholinyl, thiomorpholinyl, tetrahydrothienyl or benzodioxolanyl are particularly preferred.

Preferred compounds of Formula I are ethyl-4-chloroacetoacetate, methylacetoacetate, ethyl-8-chloro-6-oxooctanoic acid, ethyl-3-oxovaleriate, 4-hydroxy-2-butanone, ethyl-2-oxovaleriate, ethyl-2-oxo-4-phenylbutanoic acid, ethyl pyruvate, ethylphenylglyoxylate, 1-phenyl-2-propanone, 2,3-dichloroacetophenone, acetophenone, 2-octanone, 3-octanone, 2-butanone, 2,5-hexanedione, 1,4-dichloro-2-butanone, phenacyl chloride, ethyl-4-bromoacetoacetate, 1,1-dichloroacetone, 1,1,3-trichloroacetone, 1,1,1-trifluoroacetone and 1-chloroacetone.

In the process according to the invention, the enzyme can either be used in a completely or partially purified state or while being included in cells. Thereby, the cells being used can be provided in a native, permeabilized or lysed state.

10 000 to 10 Mio U of oxidoreductase are used per kg of compound of Formula I to be converted (no upper limit). Thereby, the enzyme unit 1 U corresponds to the enzyme amount which is required for reacting 1 μmol of the compound of Formula I per minute (min).

In addition to the oxidoreductase for the enantioselective keto reduction, a further oxidoreductase, preferably a secondary alcohol dehydrogenase, can also be included for the coenzyme regeneration. Suitable secondary alcohol dehydrogenases are, for example, those from Thermoanaerobium brockii, Clostridium beijerinckii, Lactobacillus minor or Lactobacillus brevis, Pichia capsulata, Candida parapsilosis, Rhodococcus erythropolis.

In the process according to the invention, the alcohol dehydrogenase can either be used in a completely or partially purified state or whole cells containing the alcohol dehydrogenase can be used. Thereby, the cells being used can be provided in a native, permeabilized or lysed state.

A buffer, e.g., a potassium phosphate, tris/HCl or triethanolarnine buffer having a pH value of from 5 to 10, preferably a pH value of from 6 to 9, can be added to the water. In addition, the buffer can contain ions for stabilizing or activating both enzymes, for example magnesium ions for stabilizing the alcohol dehydrogenase from Lactobacillus minor.

The substrate can be solid or liquid, water-soluble or water-insoluble. During the reaction, the substrate can furthermore exist in a completely or also in an incompletely dissolved state. The reaction batch can contain an additional organic solvent. Preferred organic solvents are, for example, ethyl acetate, tertiary butyl methyl ether, diisopropyl ether, heptane, hexane or cyclohexane or mixtures thereof of different composition.

The concentration of the cofactor NAD(P)H, based on the aqueous phase, ranges from 0.001 mM to 1 mM, particularly from 0.01 mM to 0.1 mM.

In the process according to the invention, the compounds of Formula I are used, for example, in an amount of from 2%-50% (w/v), based on the total volume, preferably from 10% to 30% (w/v).

The process according to the invention is carried out, for example, in a closed reaction vessel made of glass or metal. For this purpose, the components are transferred individually into the reaction vessel and stirred under an atmosphere of, e.g., nitrogen or air. Depending on the substrate and the compound of Formula I being used, the reaction time is from 1 hour to 96 hours, in particular from 2 hours to 24 hours.

Vice versa, the process according to the invention can also be employed for the enzyme-catalyzed oxidation reaction. The reaction conditions are essentially the same as in the above-mentioned process for the enantiospecific reduction of the keto compound of Formula I. However, in the process, instead of an enantioselective reduction of the keto compound of Formula I, the corresponding hydroxy compound of Formula II is oxidized to the corresponding keto compound. Furthermore, instead of 4-methyl-2-pentanol, 5-methyl-2-hexanol and 5-methyl-3-heptanol, respectively, the inexpensive corresponding ketones 4-methyl-2-pentanone, 5-methyl-2-hexanone and 5-methyl-3-heptanone, respectively, are used in the process for the regeneration of NAD(P). If a racemic hydroxy compound of Formula II is used in combination with an enantioselective oxidoreductase, the keto compound of Formula I and an enantiomer of the racemic hydroxy compound of Formula II is obtained in the course of this.

However, the process can also be employed for the preparation of poorly accessible keto compounds from the racemic alcohols thereof, using unselective oxidoreductases or also mixtures of enantioselective oxidoreductases.

Below, the invention is illustrated in further detail by examples.

### EXAMPLES

The reduction of the compounds of Formula 1 is performed by transferring the components indicated below into a reaction vessel and incubating them at room temperature as they are being thoroughly mixed.

1. Synthesis of (R)-ethyl-4-chloro-3-hydroxybutyric acid with an NADH-dependent enzyme from Candida parapsilosis

| | | | | | concen- |
|---|---|---|---|---|---|
| Component | amount | | percent | | tration |
| buffer | 1 | ml | | | |
| 100 mM triethanolamine | | | | | |
| buffer | | | | | |
| pH = 7.5; 2 mM ZnCl₂ | | | | | |
| 10% glycerol | | | | | |
| NAD [M = 663 g/mol] | 2 | mg | 3 | μmol | 0.3 mM |
| 4-methyl-2-pentanol | 7 | ml | | | |
| ethyl-4-chloroacetoacetate | 2 ml = | | 20% | (v/v) | |
| (164 g/mol, d = 1.2 g/ml) | 2.4 g | | 14.6 | mmol | |
| enzyme = | 1200 | U | | | |
| S-ADH from Candida | | | | | |
| parapsilosis | | | | | |
| volume | 10 | ml | | | |
| system | biphasic | | | | |
| coenzyme regeneration | substrate-coupled | | | | |
| incubation period | 24 | h | | | |
| conversion | >99% | | | | |
| ee-value | >99% | | | | |
| ttn NAD | 4866 | | | | |
| enzyme consumption | 500 | U/g | | | |

Upon completion of the reaction, the aqueous phase is separated from the organic phase containing the product, and the product (R)ethyl-4-chloro-3-hydroxybutyrate is purified from 4-methyl-2-pentanol by distillation. In this manner, the (R)ethyl-4-chloro-3-hydroxybutyrate can be obtained in high chemical and optical purity.

2. Synthesis of S,S-butanediol with an NADH-dependent enzyme from Candida parapsilosis

| | | | | | concen- |
|---|---|---|---|---|---|
| Component | amount | | percent | | tration |
| buffer | 1 | ml | | | |
| 100 mM triethanolamine | | | | | |
| buffer pH = 7.5; | | | | | |
| 2 mM ZnCl_{2,} 10% | | | | | |
| glycerol | | | | | |
| NAD [M = 663 g/mol] | 1 | mg | 1.5 | μmol | 0.15 mM |
| 4-methyl-2-pentanol | 8 | ml | | | |
| 4-hydroxy-2-butanone | 1 | ml | 10% | (v/v) | |
| (M = 88.12 g/mol,) | | | | | |
| enzyme = | 1000 | U | | | |
| S-ADH from Candida | | | | | |
| parapsilosis | | | | | |
| volume | 10 | ml | | | |
| system | biphasic | | | | |
| coenzyme regeneration | substrate- | | | | |
| | coupled | | | | |
| incubation period | 24 | h | | | |
| conversion | >90 | | | | |
| ee-value | >99% | | | | |
| ttn NAD | 6630 | | | | |
| enzyme consumption | 500 | U/g | | | |

Upon completion of the reaction, the aqueous phase is separated from the organic phase containing the product, and the product (S,S)-butanediol is purified from 4-metbyl-2-pentanol by distillation. In this manner, the (S,S)-butanediol can be obtained in high chemical and optical purity.

3. Synthesis of 2,5-S,S-hexanediol with an NADH-dependent enzyme from Candida parapsilosis

| | | | | | concen- |
|---|---|---|---|---|---|
| Component | amount | | percent | | tration |
| buffer | 100 | ml | | | |
| 100 mM triethanolamine | | | | | |
| buffer pH = 7.5 | | | | | |
| NAD [M = 663 g/mol] | 100 | mg | =0.15 | mmol | |
| 4-methyl-2-pentanol | 800 | ml | | | |
| 2,5-hexanedione | 100 ml = | | 10% | (v/v) | |
| (114 g/mol, d = 1) | 0.87 mol | | | | |
| enzyme = | 36 000 | U | | | |
| S-ADH from Candida | | | | | |
| parapsilosis | | | | | |
| volume | 1 | l | | | |
| system | biphasic | | | | |
| coenzyme regeneration | substrate- | | | | |
| | coupled | | | | |
| incubation period | 24 | h | | | |
| conversion | 67% | | | | |
| ee-value | >99.9 | | | | |
| ttn NAD | 5800 | | | | |
| enzyme consumption | 360 | U/g | | | |

Upon completion of the reaction, the aqueous phase is separated from the organic phase containing the product, and the product/educt mixture 2,5-(S,S)-hexanediol/2,5-hexanedione is separated from 4-methyl-2-pentanol by distillation.

The product 2,5-(S,S)-hexanediol can be separated from the educt 2,5-hexanedione in a subsequent vacuum distillation and can be obtained in a chemical purity of >99%. The total yield of the process thereby amounts to, e.g., 40-60%.

4. Synthesis of (R)-2-chloro-1-(3-chlorophenyl)ethane-1-ol with an NADH-dependent enzyme from Pichia capsulata

| | | | | | concen- |
|---|---|---|---|---|---|
| Component | amount | | percent | | tration |
| 100 mM triethanolamine | 1 | ml | | | |
| buffer pH = 7; 2 | | | | | |
| mM ZnCl₂, 10% glycerol | | | | | |
| NAD [M = 663 g/mol] | 0.5 | mg | 0.75 | μmol | |
| 4-methyl-2-pentanol | 8 | ml | | | |
| 2-chloro-1-(3- | 1 | g | 5.2 | mmol | |
| chlorophenyl)ethane- | | | | | |
| 1-one | | | | | |
| (M = 189 g/mol) | | | | | |
| enzyme = | 1000 | U | | | |
| S-ADH from Pichia | | | | | |
| capsulata | | | | | |
| (DE10327454.4) | | | | | |
| volume | 10 | ml | | | |
| system | biphasic | | | | |
| coenzyme regeneration | substrate- | | | | |
| | coupled | | | | |
| incubation period | 24 | h | | | |
| conversion | >99% | | | | |
| ee-value | >99% | | | | |
| ttn NAD | 6900 | | | | |
| enzyme consumption | 1000 | U/g | | | |

Upon completion of the reaction, the aqueous phase is separated from the organic phase containing the product, and the product 2-chloro-1-(3-chlorophenyl)ethane-1-ol is separated from 4-methyl-2-pentanol by distillation.

5. Reduction of 8-chloro-6-oxooctanoic acid ethyl ester to S-8-chloro-6-hydroxyoctanoic acid ethyl ester via NADPH-dependent oxidoreductase

| | | | | | concen- |
|---|---|---|---|---|---|
| Component | amount | | percent | | tration |
| 100 mM potassium phosphate | 2 | ml | | | |
| buffer | | | | | |
| pH = 8.5, 10% glycerol | | | | | |
| NADP [M = 765 g/mol] | 0.1 | mg | 0.13 | μmol | |
| 4-methyl-2-pentanol | 5 | ml | | | |
| S-8-chloro-6-oxo-octanoic acid | 0.5 | ml | 2.2 | mmol | |
| ethyl ester | | | | | |
| 222.71 g/mol | | | | | |
| enzyme = | 240 | U | | | |
| oxidoreductase from | | | | | |
| Lactobacillus reuteri | | | | | |
| DE 103 00 335.5 | | | | | |
| enzyme | 240 | U | | | |
| ADH from Thermoanerobium | | | | | |
| brockii | | | | | |
| volume | 8 | ml | | | |
| system | biphasic | | | | |
| coenzyme regeneration | enzyme- | | | | |
| | coupled | | | | |
| incubation period | 24 | h | | | |
| conversion | 90% | | | | |
| ee-value | 97% | | | | |
| ttn NADP | 17000 | | | | |
| enzyme consumption | 480 | U/g | | | |
| Lactobacillus reuteri | | | | | |
| enzyme consumption | 480 | U/ | | | |
| Thermoanerobium brockii | | | | | |

6. Reduction of3-oxovaleric acid methyl ester to S-3-hydroxy-oxovaleric acid methyl ester, Pichia capsulata

| | | | | concen- |
|---|---|---|---|---|
| Component | amount | | percent | tration |
| buffer | 450 | μl | | |
| 100 mM triethanolamine buffer | | | | |
| pH = 7.0 | | | | |
| 10% glycerol | | | | |
| NAD [M = 663 g/mol] | 0.1 | mg | | |
| 4-methyl-2-pentanol | 450 | μl | | |
| methyl-3-oxovaleriate | 100 | μl | | |
| enzyme = | 50 | U | | |
| S-ADH from Pichia capsulata | | | | |
| (DE10327454.4) | | | | |
| volume | 1000 | μl | | |
| system | biphasic | | | |
| coenzyme regeneration | substrate- | | | |
| | coupled | | | |
| incubation period | 24 | h | | |
| conversion | 95% | | | |
| ee-value | >99% | | | |
| ttn NAD | 5300 | | | |
| enzyme consumption | 500 | U/g | | |

1. A process for the enantioselective enzymatic reduction of keto compounds of general Formula I
R₁—C(O)—R₂ (I)
wherein R1 stands for one of the moieties
1) —(C₁—C₂₀)-alkyl, wherein alkyl is linear-chain or branched,
2) —(C₂—C₂₀)-alkenyl, wherein alkenyl is linear-chain or branched and optionally contains up to four double bonds,
3) —(C₂—C₂₀)-alkynyl, wherein alkynyl is linear-chain or branched and optionally contains up to four triple bonds,
4) —(C₆—C₁₄)-aryl,
5) —(C₁—C₈)-alkyl-(C₆—C₁₄)-aryl,
6) —(C₅—C₁₄)-heterocycle which is unsubstituted or substituted one, two or three times by —OH, halogen, —NO₂ and/or —NH₂, or
7) —(C₃—C₇)-cycloalkyl,
wherein the moieties mentioned above under 1) to 7) are unsubstituted or substituted one, two or three times, independently of each other, by —OH, halogen, —NO₂ and/or —NH₂,
and R₂ stands for one of the moieties
8) —(C₁—C₆)-alkyl, wherein alkyl is linear-chain or branched,
9) —(C₂—C₆)-alkenyl, wherein alkenyl is linear-chain or branched and optionally contains up to three double bonds,
10) —(C₂—C₆)-alkynyl, wherein alkynyl is linear-chain or branched and optionally contains two triple bonds, or
11) —(C₁—C₁₀)-alkyl-C(O)—O—(C₁—C₆)-alkyl, wherein alkyl is linear or branched and is unsubstituted or substituted one, two or three times by —OH, halogen, —NO₂ and/or —NH₂, wherein the moieties mentioned above under 8) to 11) are unsubstituted or substituted one, two or three times, independently of each other, by —OH, halogen, —NO₂ and/or —NH₂,
wherein the keto compounds are insoluble in water,
characterized in that
a liquid, two-phase mixture containing
(a) at least 5% by weight/by volume of a compound of Formula (I),
(b) at least 10% by volume of 4-methyl-2-pentanol, 5-methyl-2-hexanol and/or 2-heptanol and
(c) water,
is treated with an oxidoreductase in the presence of a cofactor in order to form a chiral hydroxy compound of general Formula II
R₁—CH(OH)—R₂ (II)
wherein R₁ and R₂ have the above-indicated meanings.

2. A process according to claim 1, characterized in that the oxidoreductase is of a microbial origin and originates in particular from bacteria of the group of Lactobacillales, particularly of the genus Lactobacillus, or from yeasts, particularly of the genera Pichia, Candida, Pachysolen, Debaromyces or Issatschenkia.

3. A process according to any of claims 1 or 2, characterized in that NAD(P)H is used as the cofactor.

4. A process according to any of claims 1 to 3, characterized in that the liquid, two-phase mixture contains at least 40% by volume of 4-methyl-2-pentanol, 5-methyl-2-hexanol and/or 2-heptanol if an oxidoreductase of a microbial origin is used.

5. A process according to claim 4, characterized in that the liquid, two-phase mixture contains between 40 and 80% by volume of 4-methyl-2-pentanol, 5-methyl-2-hexanol and/or 2-heptanol.

6. A process according to any of claims 1 to 5, characterized in that the liquid, two-phase mixture contains the compound of general Formula (I) in an amount of between 2 and 50% by weight/by volume, particularly between 10 and 50% by weight/by volume.

7. A process according to any of claims 1 to 6, characterized in that ethyl-4-chloroacetoacetate, methylacetoacetate, ethyl-8-chloro-6-oxooctanoic acid, ethyl-3-oxovaleriate, 4-hydroxy-2-butanone, ethyl-2-oxovaleriate, ethyl-2-oxo-4-phenylbutyric acid, ethylpyruvate, ethylphenylglyoxylate, 1-phenyl-2-propanone, 2,3-dichloroacetophenone, acetophenone, 2-octanone, 3-octanone, 2-butanone, 1,4-dichloro-2-butanone, phenacyl chloride, ethyl-4-bromoacetoacetate, 1,1-dichloroacetone, 1,1,3-trichloroacetone, 1,1,1- trifluoroacetone or 1-chloroacetone is used as the compound of general Formula (I).
